(19) United States
(12) Patent Application Publication
(10) Pub. No.: US 2005/0059870 A1
(43) Pub. Date: Mar. 17, 2005

(54) Processing methods and apparatus for monitoring physiological parameters using physiological characteristics present within an auditory canal

(76) Inventor: John Gregory Aceti, West Windsor, NJ (US)

Correspondence Address: RATNERPRESTIA, P.O. BOX 980, VALLEY FORGE, PA, 19482-0980, US

(21) Appl. No.: 10/925,765

(22) Filed: Aug. 25, 2004

Related U.S. Application Data
(60) Provisional application No. 60/497,890, filed on Aug. 25, 2003.

(51) Int. Cl. A61B 5/00; A61B 5/11; A61B 10/00

(52) U.S. Cl. 600/340; 600/595; 600/549; 600/551; 128/903

(57) ABSTRACT

Methods and apparatus for monitoring at least one physiological parameter of an animal from one or more physiological characteristics present within an auditory canal of the animal. Physiological parameters are measured by sensing at least one physiological characteristic present within the auditory canal of the animal, the at least one physiological characteristic associated with a physiological parameter, and processing the at least one sensed physiological characteristic at a device positioned remotely from the auditory canal to determine the physiological parameter.

### CROSS REFERENCE TO RELATED APPLICATION

This application claims the benefit of U.S. Provisional Application No. 60/497,890, filed Aug. 25, 2003, the contents of which are incorporated herein by reference.

### FIELD OF THE INVENTION

The present invention relates to methods and apparatus for monitoring physiological parameters and, more particularly, to processing methods and apparatus for monitoring physiological parameters using physiological characteristics present within an auditory canal of an animal.

### BACKGROUND OF THE INVENTION

Physiological parameters are routinely monitored in a wide range of medical applications. Instruments for use in the auditory canal to measure physiological parameters have been developed. See, for example, U.S. Pat. No. 6,283,915 to Aceti et al., entitled DISPOSABLE IN-THE-EAR MONITORING INSTRUMENT AND METHOD OF MANUFACTURER. These instruments incorporate miniaturized components for monitoring physiological parameters along with a small battery into a package that is configured for placement within the ear. Such instruments provide an unobtrusive way to monitor physiological parameters. Miniaturized components, however, are typically more expensive than larger component, and small batteries tend to have relatively short life spans.

There is an ever-present desire for less expensive medical instruments having longer battery life spans. Accordingly, improved methods and apparatus are needed for monitoring physiological parameters that are not subject to the above limitations. The present invention addresses this need among others.

### SUMMARY OF THE INVENTION

The present invention is embodied in methods and apparatus for monitoring at least one physiological parameter of an animal from one or more physiological characteristics present within an auditory canal of the animal. Physiological parameters are measured by sensing at least one physiological characteristic present within the auditory canal of the animal, the at least one physiological characteristic associated with a physiological parameter, and processing the sensed physiological characteristic at a device positioned remotely from the auditory canal to determine the physiological parameter.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that, according to common practice, the various features of the drawings are not drawn to scale. On the contrary, the dimensions of the various features are arbitrarily expanded or reduced for clarity. Included in the drawings are the following figures:

FIG. 1 depicts a partially exploded view of an exemplary monitoring device in accordance with the present invention;

FIG. 2 depicts the exemplary monitoring device of FIG. 1 positioned on the head of an animal;

FIG. 3 is a block diagram of exemplary components within the exemplary monitoring device in accordance with the present invention;

FIG. 4 is a cross-sectional view of a section of a conductor portion of the monitoring device configured for positioning within the auditory canal in accordance with the present invention;

FIG. 5 is an illustration of a sheath for covering at least a portion of a monitoring device in accordance with the present invention;

FIG. 6 is an illustration of a sheath partially positioned to cover a portion of the monitoring device in accordance with the present invention;

FIG. 7 is an illustration of a sheath fully positioned to cover a portion of the monitoring device in accordance with the present invention;

FIG. 8 is a block diagram of a monitoring system in accordance with the present invention; and

FIG. 9 is a flow chart of exemplary steps for determining physiological parameters in accordance with the present invention.

### DETAILED DESCRIPTION OF THE INVENTION

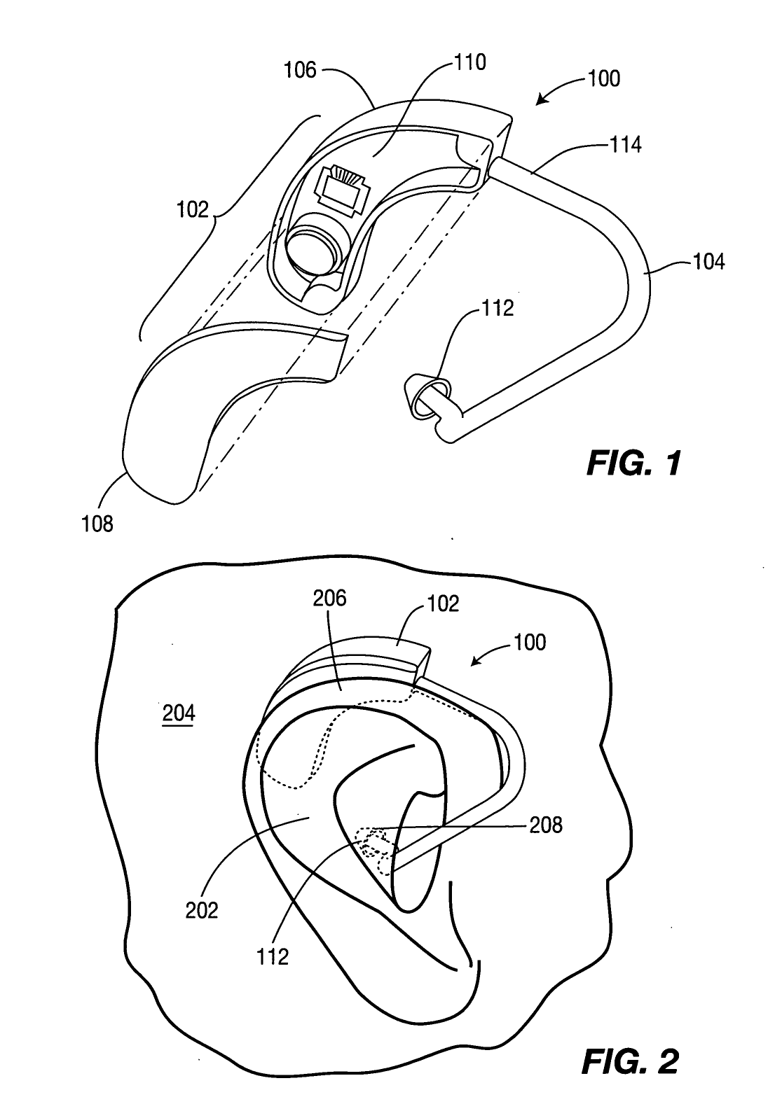

FIG. 1 and FIG. 2 are useful for providing a general overview of the present invention. FIG. 1 depicts an exemplary monitoring device 100 in accordance with the present invention. The monitoring device 100 includes a processor portion 102 and a conductor portion 104. In an exemplary embodiment, the conductor portion 104 is removably coupled to the processor portion 102 and is considered disposable.

The illustrated processor portion 102 includes a housing 106 with a cover 108 removed therefrom to expose electrical and/or electronic components 110 contained therein. Additionally, electrical and/or electronic components 110 may be found within the conductor portion 104. The conductor portion 104 includes a first end 112 configured for insertion at least partially within the auditory canal of an animal and a second end 114 coupled to the processor portion 102.

In use, the first end 112 of the conductor portion is positioned at least partially within the auditory canal of the animal to detect one or more physiological characteristics and pass the detected physiological characteristics through the conductor portion 104 from the first end 112 to the second end 114 for processing by the processor portion 102 to determine at least one physiological parameter. The one or more physiological characteristics are associated with the at least one physiological parameter and include, by way of non-limiting example, temperature, light intensity, and sound. The associated physiological parameters include, by way of non-limiting example, temperature, pulse, blood-oxygen content, and respiration rate. For example, the intensity of light transmitted through tissue of an auditory canal wall may be used in accordance with known pulse-oximetry techniques to determine pulse rate and blood-oxygen content. In addition, sounds within the auditory canal may be used to determine pulse and/or respiration rate. One or more physiological characteristics such as temperature may be considered both a physiological characteristic and a physiological parameter. Other suitable physiological characteristics and parameters will be understood by those of skill in the art from the description herein.

FIG. 2 depicts the exemplary monitoring device 100 positioned relative to an ear 202 on the head 204 of an animal. The ear 202 includes an auricle 206 and an auditory canal 208 adjacent the auricle 206. In an exemplary embodiment, the processor portion 102 of the monitoring device 100 is positioned at least partially between the auricle 206 and the head 204 of the animal and the first end 112 of the conductor portion 104 is positioned at least partially within the auditory canal 208. In an alternative exemplary embodiment, the processor portion 102 may be positioned in essentially any location remote to the auditory canal. The animal may be a human being, a domestic animal such as a cow, horse, dog, or cat, a wild animal such as a lion or elephant, or essentially any animal having an ear with an auditory canal.

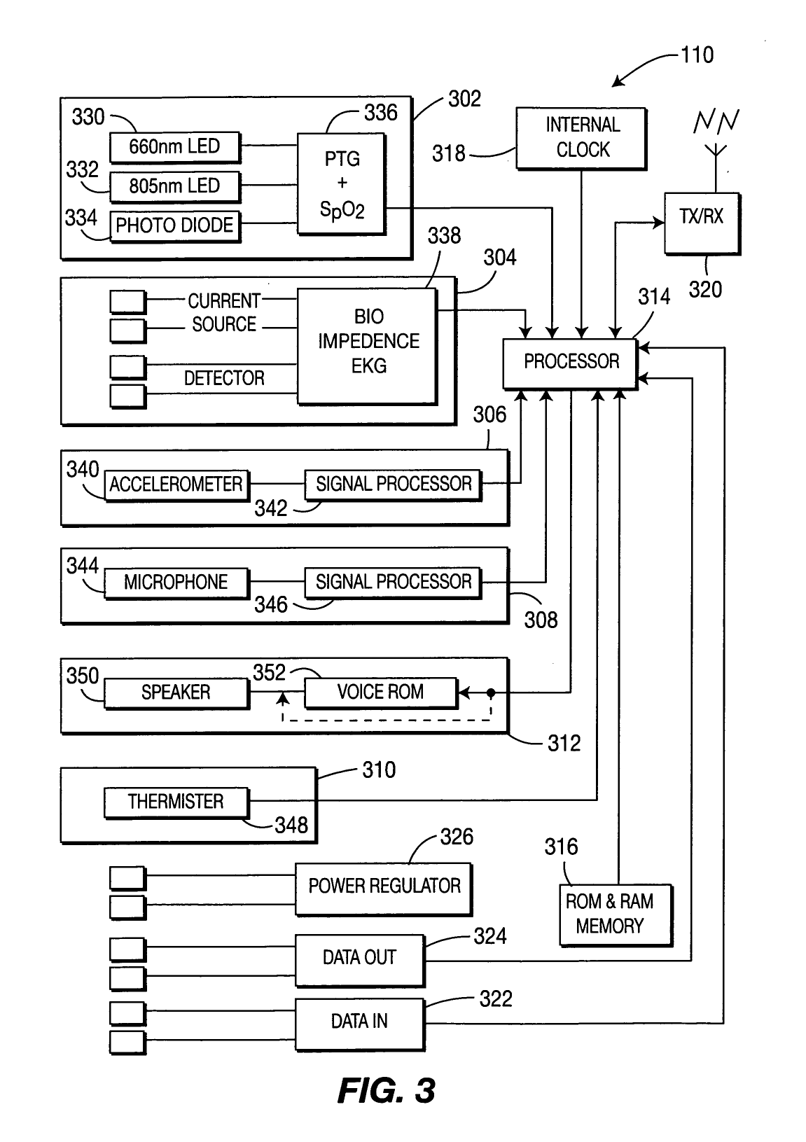

The present invention is now described in detail. FIG. 3 depicts exemplary electrical and/or electronic components 110 (referred to herein as components 110) that may be located within the monitoring device 100 (FIG. 1). The illustrated components 110, which are described below with reference to FIGS. 1 and 2, include a presentation device 312 (e.g., a speaker 350 and, optionally, a voice read only memory (ROM) 352), a memory 316, an internal clock 318, a transceiver 320 (or, optionally, a transmitter only), data input circuitry 322, data output circuitry 324, and one or more sensors (i.e., five in the illustrated embodiment). The illustrated sensors include a pulse oximetry sensor 302, an electrocardiogram sensor 304, an accelerometer 306, a microphone 308, and a thermister 320, each of which will be described in further detail below.

A processor 314 is configured to process signals from the sensors, present information (e.g., via the presentation device 312), and communicate information (e.g., via the data input/output circuitry 322/324 and the transceiver 320). Further, the processor 314 is configured to store information to the memory 316 and retrieve the information from the memory 316. The internal clock 318 provides the processor with real time and/or interval readings for use in processing the information from the sensors. A power regulator 326 is optionally included to regulate power to the electrical and/or electronic components 110. A suitable processor 314, memory 316, internal clock 318, transceiver 320, data input circuitry 322, data output circuitry 324, and power regulator 326 will be understood by those of skill in the art from the description herein.

One or more of the sensors may reside in the conductor portion 104 near the first end 112 to sense physiological characteristics within the auditory canal. In this embodiment, the sensors sense the physiological characteristics and generate electrical signals that are passed through the conductor portion to the processor 314 in the processor portion 102, e.g., via an electrically conductive wire (referred to here as a wire). Alternatively, one or more of the sensors may be positioned within the processor portion 102 with physiological characteristics within the auditory canal being passed through the conductor portion 104, e.g., via acoustic tubes, fiber optic cables, or wires, as described in further detail below.

Acoustic tubes communicate aural signals through the conductor portion 104 between the auditory canal and the processor portion 102. Acoustic tubes may be used to transfer sounds from the auditory canal, such as those due to respiration, to the processor portion 102 and/or to transfer aural messages from a speaker 350 in the processor portion 102 to the auditory canal. Those of skill in the art of hearing aids have developed various tube configurations for delivering sound to the auditory canal. Such tubes can also be used for receiving sounds from the auditory canal.

Fiber optic cables communicate photonic signals through the conductor portion 104 between the auditory canal and the processor portion 102. Fiber optic cables may be used to transfer one or more wavelengths of light generated in the processor portion 102 to the auditory canal and to transfer one or more wavelengths of light in the auditory canal (e.g., emanating from the auditory canal wall tissue) to the processor portion 102.

Wires communicate electric/electronic signals through the conductor portion 104 between the auditory canal and the processor portion 102. Wires may be used to transfer electric/electronic signals generated in the processor portion 102 to the auditory canal or a sensor within the conductor portion 104 positioned in the auditory canal and to transfer electric/electronic signals in the auditory canal (e.g., emanating from the auditory canal wall tissue or a sensor within the conductor portion 104 positioned in the auditory canal) to the processor portion 102. The wires may terminate with electrodes suitable for contact with auditory canal wall tissue. In an exemplary embodiment, the electrodes are mounted in an ear mold, which is described in further detail below.

In an exemplary embodiment, the conductor portion 104 and the wires, acoustic tubes, and/or fiber optic cables extending through the conductor portion 104 are flexible and/or moldable. This enables sensors within the conductor portion 104 to be at least partially mechanically separated from the processing portion 102 to prevent/reduce the transfer of motion of the processing device 102 to the sensors within the conductor portion 104, which could cause erroneous signals. In addition, this enables the conductor portion 104 to conform to the shape of the auditory canal, thereby improving comfort.

The sensors are now described in detail. The illustrated pulse oximetry sensor 302 includes a first light emitting diode 330, a second light emitting diode 332, a photo detector diode 334, and pulse oximetry circuitry 336. For pulse oximetry, light from the first and second diodes 330 and 332 are introduced to the tissue lining the auditory canal wall in the vicinity of the first end 112 of the conductor portion 104. The photo detector diode 334 detects light (i.e., a physiological characteristic) that passes through the tissue that was introduced by the light emitting diodes 330 and 332. The pulse oximetry circuitry 336 monitors the pulses of light introduced by the LEDs 330 and 332 and the light received at the photo detector diode 334 to determine pulse rate and/or blood oxygenation levels (i.e., physiological parameters). In an exemplary embodiment, the pulse oximetry circuitry 336 may be positioned within the processor portion 102 and is connected via wires to the LEDs 330/332 and the photo diode 334, which are positioned within the first end 112 of the conductor portion 104. In an alternative exemplary embodiment, the LEDs 330/332 and/or the photo detector diode 334 may be positioned within the processor portion 102 with light from the LEDs 330 and 332 and/or light detected by the photo diode 334 being passed therebetween via fiber optic cables extending through the conductor portion 104. The pulse oximetry circuitry 336 communicates pulse oximetry information to the processor 314 for processing in a manner that will be understood by one of skill in the art from the description herein.

The electrocardiogram sensor 304 includes electrocardiogram circuitry 338 that acts as a current source and current detector. In an exemplary embodiment, the electrocardiogram circuitry 338 may be positioned within the processor portion 102 with wires leading from the processor portion 102 through the conductor portion from the second end 114 to the first end 112 where the wires contact tissue of the auditory canal wall. In an alternative exemplary embodiment, the electrocardiogram circuitry 338 may be positioned in the vicinity of the first end 112 and communicates signals via an electrical connection to the processor 314 in the processor portion 102.

The accelerometer 306 detects motion of the monitoring device 100. In an exemplary embodiment, the accelerometer 306 may be positioned within the processor portion 102. In an alternative exemplary embodiment, the accelerometer 306 may be positioned within the conductor portion 104, e.g., near the first end 112, with signals from the accelerometer 306 passed to the processor portion 102 via a wire extending through the conductor portion 104. Signal processing circuitry 342 may process signals from the accelerometer 306 into signals suitable for processing by the processor 314.

The microphone sensor 308 senses sound within the auditory canal. The microphone sensor 308 includes a microphone 344 and a signal processor 346. In an exemplary embodiment, the microphone 344 may be positioned in the processor portion 102 with audio signals from the microphone 344 being communicated from the auditory canal to the processor portion 102 through the conductor portion 104 via an acoustic tube. The acoustic tube may be sized to enable passage of the voice communication band, e.g., 2 mm or more in diameter. In an alternative exemplary embodiment, the microphone 344 may be positioned within the conductor portion 104, e.g., near the first end 112 and electrical signals generated by the microphone 344 are communicated to the processor portion 102 via a wire extending through the conductor portion 104.

The thermister sensor 310 senses temperature. In an exemplary embodiment, the thermister sensor 310 includes a thermister 348. The thermister 348 may be positioned within the first end 112 of the conductor portion 104. Electrical signals generated by the thermister in response to temperature within the auditory canal at the first end 112 may be communicated to the processor portion 102 via a wire extending through the conductor portion 104. In alternative exemplary embodiments, other devices for sensing temperature such as a thermopile may be employed to sense temperature.

The presentation device 312 presents audio signals within the auditory canal. The presentation device includes a speaker 350 and an optional voice ROM 352. In an exemplary embodiment, the speaker 350 may be positioned within the processor portion 102 with audio signals presented by the speaker 350 being communicated to the auditory canal via an acoustic tube. In an alternative exemplary embodiment, the speaker 350 may be positioned within the conductor portion 104, e.g., near the first end 112, with electrical/electronic signals being communicated from the processor portion 102 to the speaker 350 for conversion to audio signals via a wire extending through the conductor portion 104. The voice ROM 353 may store predefined messages for presentation via the speaker 350 in response to signals received from the processor 314.

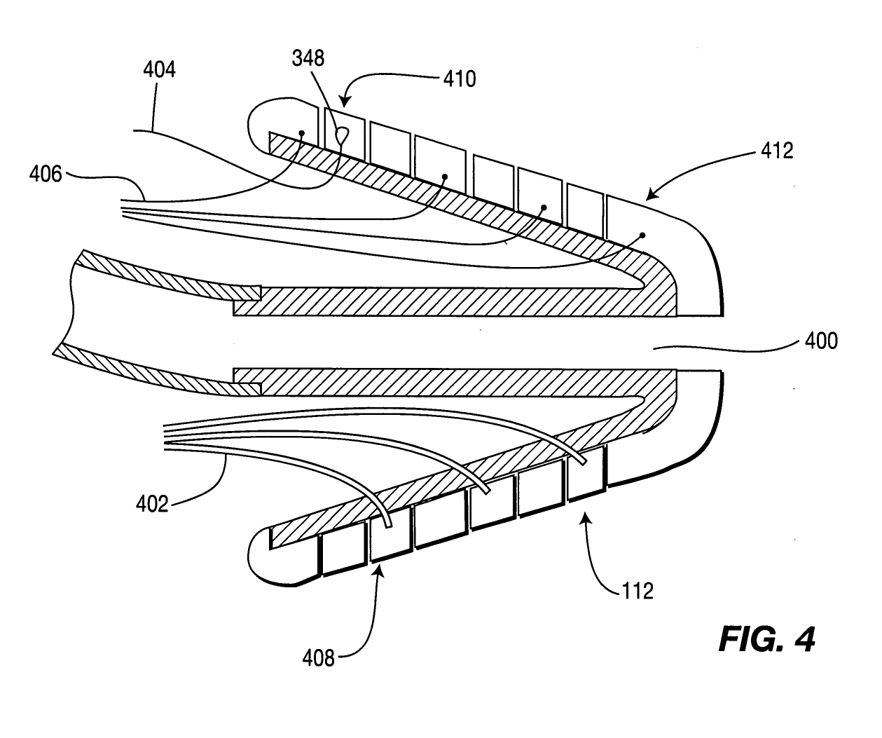

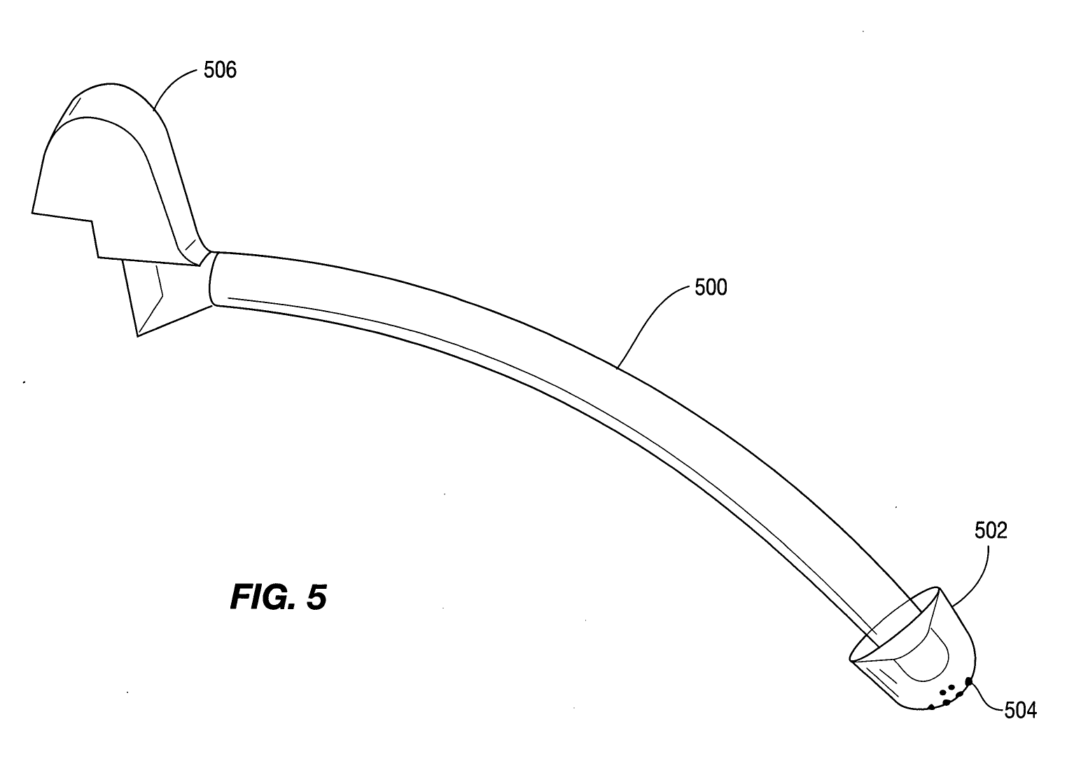

FIG. 4 depicts an exemplary embodiment of a section of the first end 112 of the conductor portion 104. The illustrated first end 112 includes an acoustic tube 400, fiber optic cables (represented by fiber optic cable 402), and wires (represented by a first electrical wire 404 and a second electrical wire 406). In the illustrated embodiment, the acoustic tube 400 extends through the center of the first end 112. In an exemplary embodiment, the acoustic tube 400 extends through the conductor portion 104 to the processor portion 102 coupled to the second end 114 (FIG. 1) of the conductor portion 104 (FIG. 1). The fiber optic cable 402 terminates in an optically transparent elastomer of the first end 112 to allow the communication of light between the fiber optic cable 402 and the tissue of the auditory canal wall. The first electrical wire 404 may be coupled to a thermister 348 embedded within a thermally conductive elastomer 410, which allows the communication of temperature from the auditory canal wall tissue to the thermister 348. The second electrical wire 406 terminates in an electrically conductive elastomer 412, which allows the communication of electrical signals to/from the auditory canal wall tissue. In an exemplary embodiment, the first end 112 may be sized such that when inserted within the auditory canal, the outer surface of the first end 112 (e.g., the optically transparent elastomer 408), the thermally conducting elastomer 410, and the electrically conducting elastomer 412 contact the wall of the auditory canal. In an exemplary embodiment, the first end 112 is configured for comfort, biocompatibility, durability, and ease of manufacture. Suitable materials for use within the first end 112 include acrylic, vinyl, silicone, or polyethylene, for example.

In an exemplary embodiment, the processor portion 102 (FIG. 1) includes a power source (not shown), sensors (except for the thermister 348), an RF transceiver 320, and connection means (not shown) for connection to the electrical wires 406/408, acoustic tube 400, and fiber optic cables 402. In accordance with this embodiment, the conductor portion 104 includes the thermister 348, electrical wires 406/408, acoustic tube 400, and fiber optic cables 402, and provides structural support therefore. This embodiment minimizes the cost of the conductor portion 104, making the conductor portion disposable.

The monitoring device 100 provides, by way of non-limiting example, enhanced comfort for some animals over devices positioned entirely within the auditory canal, better fit for a larger percentage of animals, easy configuration for extreme auditory canal sizes or shapes. Further, due to its larger size (as compared to a monitoring device that is designed to fit entirely within the auditory canal), the monitoring device 100 provides greater flexibility in battery selection (and, thus, battery life span), easier handling, and improved component selection. For example, the larger size allows more “off-the-shelf” components to be utilized, thereby reducing potential component and development cost.

FIG. 5 depicts a flexible sheath 500 that may be used to cover at least a portion of the conductor portion 104 (FIG. 1). The flexible sheath 500 includes a tip 502 that is configured for insertion within the auditory canal and is sized to engage the auditory canal. It is contemplated that different flexible sheaths 500 with tips having various diameters, e.g., from 5 mm to 12 mm, may be provided to accommodate different auditory canal sizes. In an exemplary embodiment, the tip 502 may be acoustically, thermally, and/or optically transparent (either partially or completely). The tip may be acoustically, thermally, and/or optically transparent through the presence of holes (represented by hole 504) in the tip 502, the material of the tip, and/or the thickness of the material of the tip. In an exemplary embodiment, the holes 504 are sized to prevent cumen from entering the tip portion 502 and coming in contact with the conductor portion 104. The use of the flexible sheath 500 enables reuse of the processor portion 102 and the conductor portion 104 with the flexible sheath 500 being disposed when using the monitoring device 100 (FIG. 1) with subsequent patients or at periodic intervals with the same patient.

In an exemplary embodiment, the flexible sheath 500 is coupled to an integrated battery 506. Integrating the battery 506 into the flexible sheath provides a fresh battery for supplying power to the processor portion 102 whenever the flexible sheath 500 is exchanged.

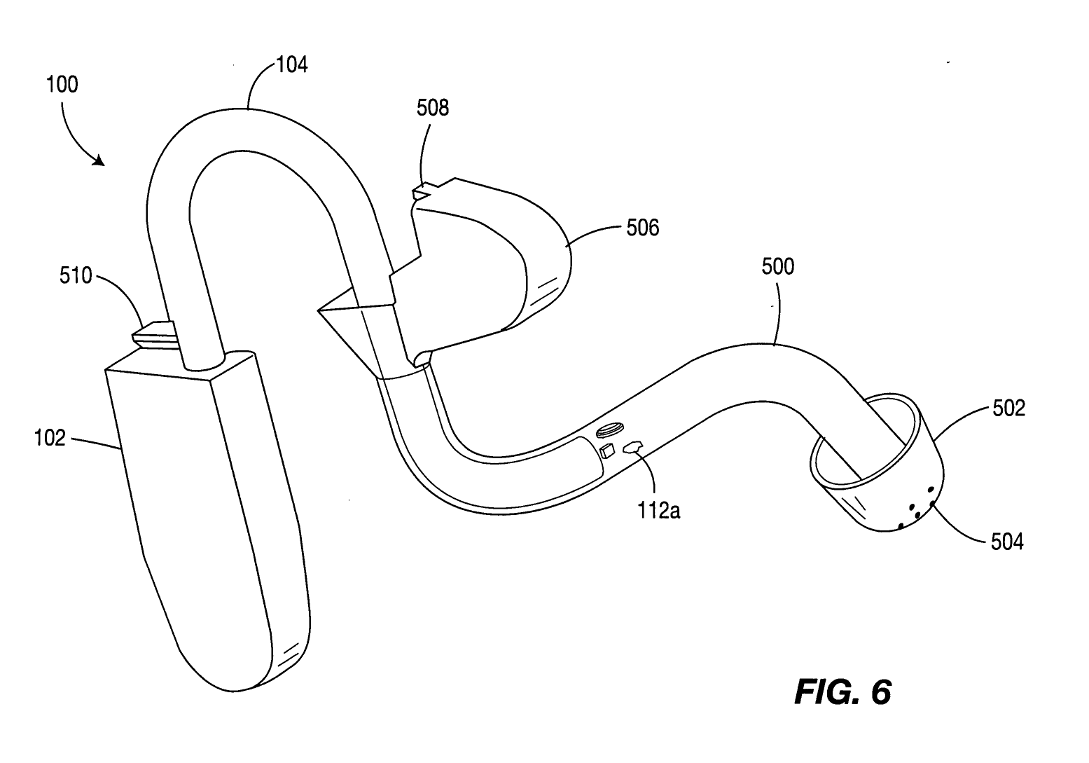

FIG. 6 depicts a monitoring device 100 with the sheath 500 partially positioned on the conductor portion 104. The monitoring device 100 illustrated in FIG. 6 includes an alternative exemplary first end 112a configured for positioning at least partially within the tip 502 of the sheath 500. In an exemplary embodiment, the first end 112a may include a speaker, microphone, thermister, light emitter(s) and/or light detector(s) (and/or wires, fiber optic cables and/or acoustic tubes for coupling to such components positioned in the processor portion 102). As seen in FIG. 6, the first end 112a of the conductor portion 104 has a diameter that is smaller than the diameter of the tip 502. In this embodiment, the tip 502 of the flexible sheath 500 may center the first end 112a within the auditory canal. In an alternative exemplary embodiment, a first end 112 such as depicted in FIG. 4 may be used with the first end 112 deforming to fit the body of the sheath 500 as the sheath is positioned on the monitoring device 100 and expanding within the tip 502 of the sheath 500 to contact the wall of the auditory canal through the tip 502 of the sheath 500 when fully positioned on the monitoring device 100. In another alternative exemplary embodiment, the body of the sheath 500 may expand to accommodate the first end 112 as the sheath 500 is positioned on the monitoring device 100 and the first end 112 may contact the wall of the auditory canal through the tip 504 of the sheath 500 when the sheath 500 is fully positioned on the monitoring device 100. Various alternative embodiments will be understood by those of skill in the art from the description herein. In an exemplary embodiment, the integrated battery 506 includes a fastener 508 for engaging a corresponding fastener 510 on the processor portion 102.

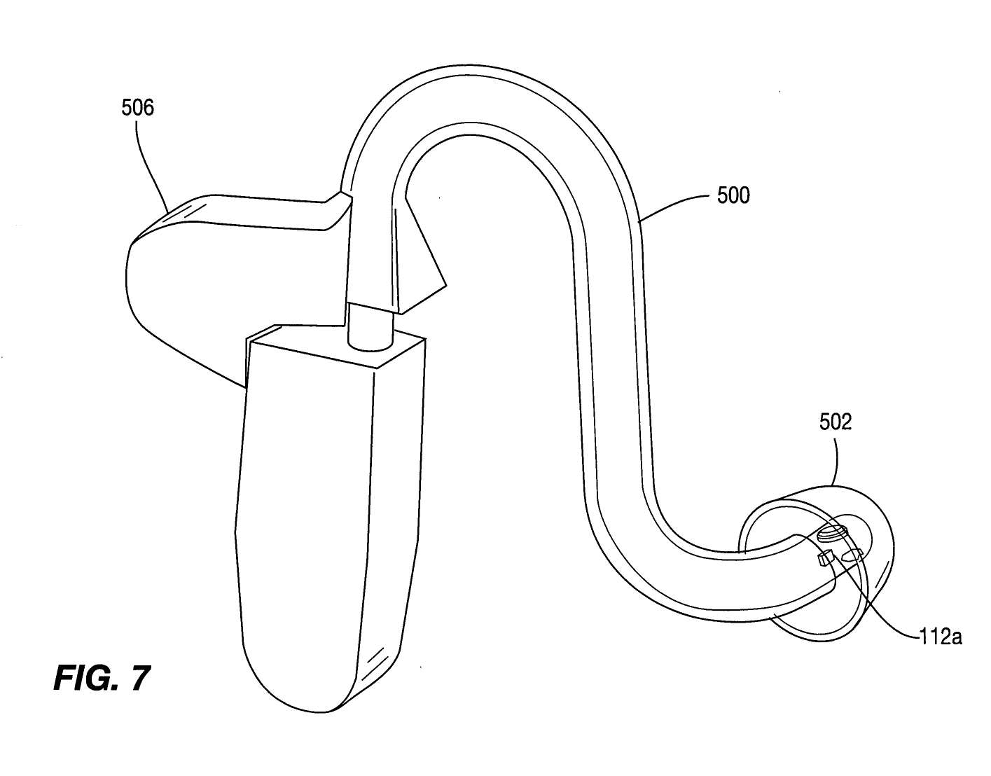

FIG. 7 depicts a fully assembled monitoring device 100 with flexible sheath installed. In an exemplary embodiment, when monitoring a new patient, the battery and flexible sheath assembly may be removed from the monitoring device and a new flexible sheath and battery assembly may be reattached to the monitoring device 100 in a single step.

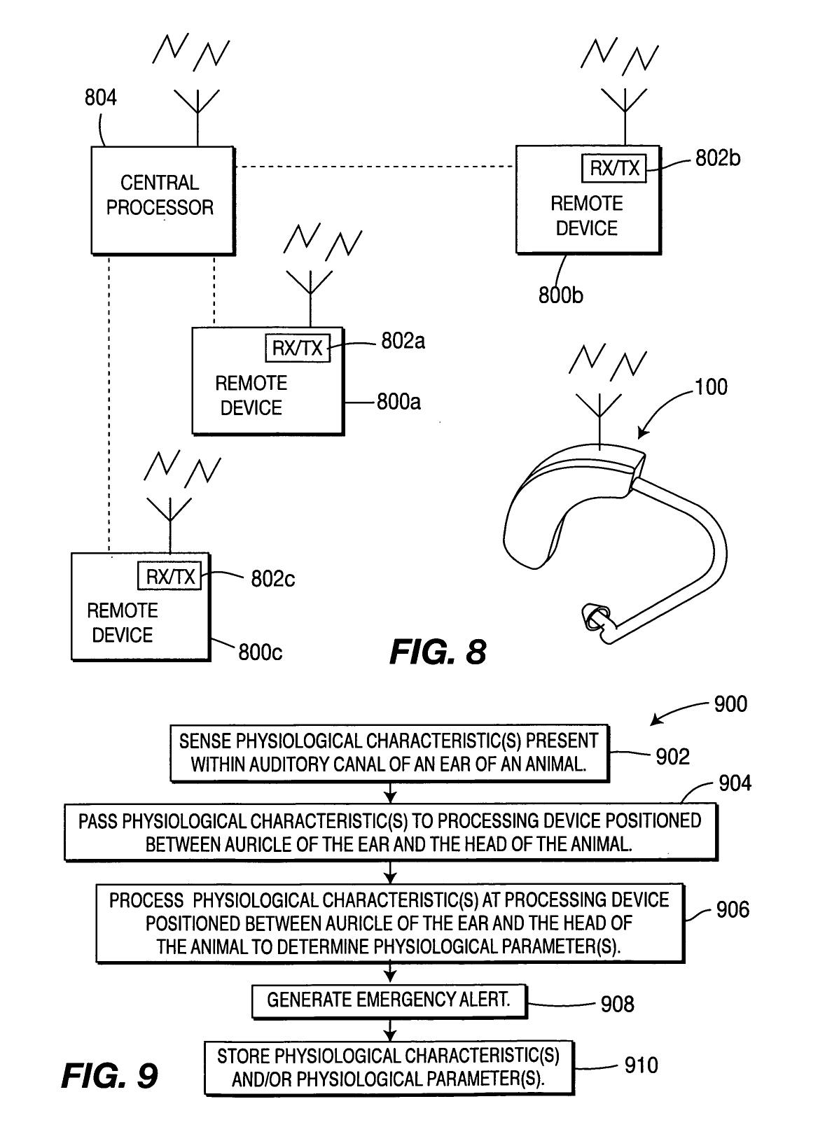

FIG. 8 depicts a monitoring device 100 and one or more remote devices (represented by remote devices 800a, b, and c). Each remote device 800 includes a transceiver (represented by transceivers 802a, b, and c) for communicating with the monitoring device 100 via the transceiver 320 (FIG. 3) of the monitoring device 100. The monitoring device 100 may communicate with one or more of the remote devices 800. The monitoring device 100 may attach an identification code to each communication with the remote devices 800 so that a particular monitoring device 100 is distinguishable from other monitoring devices. In addition, each remote device 800 may attach a unique monitoring code to communications communicated from the monitoring device 100 through the remote devices 800 to a central processing device 804 in order to provide an indication of the remote device 800 through which the monitored information was received.

FIG. 9 depicts a flow chart 900 of exemplary steps for monitoring physiological parameters in accordance with the present invention. The exemplary steps are be described with reference to FIGS. 1, 2, and 3. Physiological parameters may be monitored from one or more physiological characteristics present with an auditory canal of an animal.

At block 902, the monitoring device 100 senses one or more physiological characteristics present within the auditory canal of the animal. In an exemplary embodiment, sensors within the monitoring device 100 such as a pulse oximetry sensor 302, EKG sensor 304, accelerometer 306, microphone 308, and thermister 310 sense the one or more physiological characteristics. The sensors may be located in the processing portion 102 and/or the conductor portion 104 of the monitoring device.

At block 904, the physiological characteristics are passed from within the auditory canal to a processing device 102 positioned remote to the auditory canal, e.g., at least partially between the auricle of the ear and the head of the animal for processing. In an exemplary embodiment, the physiological characteristics may be sensed by sensors positioned in a conductor portion 104 of the monitoring device that is coupled to the processing device 102. Electrical signals representing the physiological characteristics may be generated by the sensors in the conductor portion 104 and may be communicated to the processing portion 102 for processing by the processor 314 via wires extending through the conductor portion 104.

In an alternative exemplary embodiment, physiological characteristics present within the auditory canal may be passed directly to sensors within the processing device 102 for sensing, e.g., via wires, fiber optical cables, and/or acoustic tubes. In accordance with this embodiment, the step of block 904 is performed before the step of block 902. More specifically, the physiological characteristics are passed from within the auditory canal to the processing device 102 positioned at least partially between the auricle of the ear where these physiological characteristics are then sensed.

At block 906, the sensed physiological characteristics are processed at the processing portion 102 to determine the at least one physiological parameter. In an exemplary embodiment, the processor 314 processes the physiological characteristics. In an alternative exemplary embodiment, circuitry associated with the sensors performs the processing or assists in processing the physiological characteristics.

Optionally, at block 908, an emergency alert is generated. In an exemplary embodiment, the processor 314 generates an emergency alert if a physiological characteristic or parameter is outside of a predefined range. The emergency alert may be communicated to the user wearing the monitoring device, e.g., by the processor 314 via the speaker 350 (optionally playing a predetermine message stored in the voice ROM 352). Alternatively, the emergency alert may be communicated by the processor 314 to a remote device 800 or central processing device 804 via the transceiver 320. In an alternative exemplary embodiment, the emergency alert may be generated if the monitoring device is out of communication range with a remote device 800 or a central processing device 804, or is greater than a predefined distance from these devices 800/804. In another alternative exemplary embodiment, the remote device 800 or central processing device 804 may generate the emergency alert responsive to physiological characteristics of parameters communicated from the monitoring device 100.

Optionally, at block 910, at least one of the one or more physiological characteristics or the at least one physiological parameter are stored. In an exemplary embodiment, the physiological characteristics and/or parameters are stored by the processor 314 in the memory 316. In an alternative exemplary embodiment, the physiological characteristics and/or parameters are transferred by the processor 314 (e.g., via a wired or wireless connection) to a remote device 800 (FIG. 8) or a central processing device 804 (FIG. 8) for storage.

The monitoring device 100 of the present invention has numerous novel applications. These applications include, by way of non-limiting example, location monitoring, fertility monitoring/ovulation detection, home bound patient monitoring, hospital patient monitoring, sleep apnea monitoring, Alzheimer patient monitoring, fitness monitoring, military monitoring, and emergency alert functionality. Although the monitoring device 100 described above includes a conductor portion configured for positioning at least partially within an auditory canal and a processor portion coupled to the conductor portion that is configured for positioning remote to the auditory canal, the exemplary applications may also be performed with other types of auditory canal monitoring devices that incorporate one or more of the above-described electrical and/or electronic components 110 (FIG. 3). For example, monitoring devices having a single portion or multiple portion configuration that are designed to fit at least partially within the auditory canal may be employed to perform the exemplary applications.

Location monitoring, home bound patient monitoring and hospital patient monitoring can be performed using the present invention. In an exemplary embodiment, one or more remote devices 800 (FIG. 8) may be deployed as one or more nodes (e.g., rooms) within a facility (e.g., home, hospital, care facility). Each node 800 within the facility can receive, from the monitoring device 100, emergency alerts, physiological characteristics and/or physiological parameters for processing and/or routing to a central processing device 804. In an exemplary embodiment, each node 800 may be associated with a known location such as a room number. When a node receives a communication from a monitoring device 100, the communication is tagged with the unique identification code of that particular node. The communication may then be forwarded with the node's unique identification code to the central processing device 804. At the central processing device 804, the communication may be displayed along with the location/room number, which may be deciphered by the central processing device 804 from the unique identification codes accompanying the communication.

In an alternative exemplary embodiment, signals between the transceiver 320 within the monitoring device 100 and a transceiver 802 within a remote device 800 may be monitored. The location of the patient may be determined based on signal strength, e.g., as described in U.S. Pat. No. 6,075,443 entitled WIRELESS TETHER which is commonly assigned with the present invention.

In an alternative exemplary embodiment, a user wearing the monitoring device 100 may be notified, e.g., via the speaker, that they are leaving the communication range of the remote device 800. For example, if long term data storage is maintained in the monitoring device (e.g., in the memory 316), users may be notified when they are out of communication range to prevent data loss if the monitoring device loses power. Pre-recorded warning messages may be stored within the monitoring device 100 (e.g., within the voice ROM 352). The processor 314 within the monitoring device 100 can be programmed to alert the user on a periodic basis that communication has not been restored. In addition, a care provider can be notified when communication is lost. For example, if an Alzheimer patient is leaving the vicinity of a remote device 800, the care provider is notified. In addition, the Alzheimer patient may be notified (e.g., via the voice ROM 352 and the speaker 350 within the monitoring device 100) to go to a predefined location to reestablish communication.

Fitness and exercise monitoring can be accomplished with the present invention. People of all ages can improve their health and overall quality of life with regular physical activity. The USDA Human Nutrition Research Center on Aging (HNRCA) has demonstrated that the body's decline is due to a combination of inactivity, poor nutrition, and disease. The HNRCA has identified ten key physiological factors associated with extending vitality. These factors inlcude muscle mass, strength, basal metabolic rate, body fat percentage, aerobic capacity, blood pressure, insulin sensitivity, cholesterol/HDL ratio, bone density, body temperature. The present invention enables monitoring of several of these physiological factors using the monitoring device 100 and information gathered by the monitoring device 100 can be used to assist exercise physiologists, sports trainers, and individuals in recording exercise intensities, identifying current levels of fitness, documenting performance and fitness training programs, avoiding over training, and tracking health conditions.

Ovulation detection can be accomplished with the present invention. In an exemplary embodiment, ovulation detection may be performed by monitoring temperature automatically at predetermined intervals within the auditory canal using the monitoring device 100 of the present invention. The temperature may be monitored for a predetermined period of time to develop a basal body temperature chart for monitoring the duration of the different phases of the menstrual cycle to determine if and when ovulation has occurred. Conventionally, temperature is taken and recorded manually to develop the basal body temperature chart, which is a painstaking and inefficient process. Further, increased body temperature is difficult to detect because body temperature varies up to one (1) degree Fahrenheit during the day but a change of 0.5 degrees predicates the onset of ovulation. Monitoring temperature using the monitoring device 100, however, is unobtrusive, automatic, and potentially more sensitive. In an exemplary embodiment, the accelerometer 306 measures movement such as when the user wakes up in the morning and the ovulation monitoring is further based on the detected movement.

Fall prevention monitoring (e.g., in post surgical situations) can be performed using the present invention. Frequently, patients emerging from anesthesia have an “anesthesia hangover.” Post anesthesia patients often attempt to move from a bed they are in, e.g., to go to the bathroom. Once standing, the patients may lose their balance and fall. Patients cannot be restrained and, therefore, require continuous surveillance to prevent these types of falls, which is expensive. The monitoring device 100 in accordance with the present invention can detect inclination and activity (i.e., via the accelerometer 340) and therefore electronically differentiate sleep (e.g., indicated by a supine orientation) from wakefulness (e.g., indicated by a raised orientation and motion). In a care facility, the movement of a patient can be automatically detected and an alert to a nurse located in a central monitoring station can be provided if the processor 314 determines that the movement exceeds a predefined value to assure the patient is not attempting to get out of bed. Thus, constant physical surveillance is not needed, which reduces the cost of caring for post anesthesia patients. In addition, pre-recorded alert messages may be stored within the monitoring device 100 (e.g., within the voice ROM 352) for presentation to the patient if the movement exceeds a predefined value. For example, if the monitoring device detects movement that exceeds the predefined threshold, the monitoring device 100 may aurally present an alert message to the patient, e.g., “please lay down until an assistant is available to help you.”

Sleep apnea detection may be performed using the present invention. Sleep apnea is a condition during sleep that causes air passages to become occluded—resulting in frequent sleep interruptions. Conventionally, sleep apnea detection is performed in a “sleep laboratory” where a number of vital signs, such as EEG, blood oxygen content, respiratory rate, respiratory quality, and head motion, are measured during a night of sleep. Often, a person suffering from sleep apnea has difficulty falling asleep under these conditions. Through the use of the monitoring device 100 of the present invention, the necessary vital signs can be monitored in a non-intrusive manner that permits the determination of the vital signs in laboratory and non-laboratory settings such as the home of the person. The monitoring device 100, by way of non-limiting example, monitors one or more of the following: blood oxygen content, respiratory rate, and head motion. Blood oxygen content is highly correlated with the severity of the sleep apnea due to the cyclic depression of blood oxygen as the person experiences repeated cycles of oxygen deprivation. Head motion is indicative of the frequently violent head motion that occurs when the body inhales a large amount of air after an apnea attack. Respiratory rate and quality enhance diagnosis by determining interrupted inhalation and frequency of deep breaths.

Further, therapeutics for sleep apnea include continuously forcing air into the nasal passages using a continuous positive pressure device (CPAP). The monitoring device of the present invention can provide feedback to the CPAP device to adjust flow rate, pressure, and frequency to make treatment more comfortable.

Soldier monitoring may be performed using the present invention. Soldier health and performance can deteriorate in adverse climates and situations. The success of an operation conducted under extreme environmental conditions depends upon the physical state of the individual soldiers. Dehydration and exhaustion are two factors that may lead to decreased cognitive function and, thus, adversely affect the success of the mission. The monitoring device of the present invention can provide military personnel such as commanders and medics with key physiological parameter for the individual soldiers to determine by way of non-limiting example, wounded soldiers, alive/dead status (e.g., based on heart rate), respiratory distress, thermal stress, and sleep status. The physiological parameters enable commanders to ensure that the soldiers do not become fatigued and medics to quickly identify, locate, and treat injured soldiers.

Emergency alerts may be sent using the present invention. Through the use of the monitoring device 100 including a transmitter (or transceiver) and a remote device including a receiver (or transceiver) physiological parameters outside of a normal range can automatically trigger an emergency alert. In an exemplary embodiment, a switch (not shown) on the monitoring device 100 provides immediate communication of an emergency requiring attention. If a care provider is near the remote device 800 or central processing device 804, an audible alarm alerts the care provider. If the care provider is remote to the remote device 800 or central processing device 804, the remote device 800 or central processing device 804 can automatically contact the care provider, e.g., via telephone, cellular telephone, a global network (e.g., the Internet), and/or mobile radio.

It is contemplated that one or more method steps in accordance with the invention may be implemented in software. The software may be embodied in a computer readable carrier, for example, a magnetic or optical disk, a memory-card or an audio frequency, radio-frequency, or optical carrier wave.

Although the invention is illustrated and described herein with reference to specific embodiments, the invention is not intended to be limited to the details shown. Rather, various modifications may be made in the details within the scope and range of equivalents of the claims and without departing from the invention.

1. An apparatus for monitoring at least one physiological parameter of an animal from one or more physiological characteristics present within an auditory canal of the animal, the animal having a head with an ear, the ear including the auditory canal, the apparatus comprising:
a conductor portion having a first end and a second end, the first end configured for positioning within the auditory canal of the animal, the conductor portion configured to conduct one or more physiological characteristics present within the auditory canal from the first end to the second end when the first end is positioned within the auditory canal;
a processor portion coupled to the second end of the conductor portion, the processor portion configured to receive the one or more physiological characteristics from the second end of the conductor portion and to process the one or more received physiological characteristics to monitor at least one physiological parameter.

2. The apparatus of claim 1, wherein the ear further includes an auricle adjacent the auditory canal and wherein the processor portion is configured for placement at least partially between the auricle of the ear and the head of the animal.

3. The apparatus of claim 1, wherein the conductor portion is disposable and is removably coupled to the processor portion.

4. The apparatus of claim 1, wherein the conductor portion is at least one of (i) flexible or (ii) moldable.

5. The apparatus of claim 1, wherein the first end of the conductor portion includes at least one sensor that senses at least one of the one or more physiological characteristics and generates electrical signals corresponding to the at least one sensed physiological characteristic; and
wherein the conductor portion further comprises a wire extending between the first and second ends to conduct the at least one sensed physiological characteristic from the first end to the second end as the generated electrical signal.

6. The apparatus of claim 1, wherein the conductor portion further comprises:
a wire extending between the first end and the second end to conduct one or more electrical signals.

7. The apparatus of claim 1, wherein the conductor portion further comprises:
an acoustic tube extending between the first end and the second end.

8. The apparatus of claim 7, wherein the processor portion further comprises a microphone coupled to the acoustic cable.

9. The apparatus of claim 7, wherein the processor portion further comprises a speaker coupled to the acoustic tube.

10. The apparatus of claim 1, wherein the conductor portion further comprises:
a fiber optic cable extending between the first end and the second end.

11. The apparatus of claim 10, wherein the processor portion further comprises:
an oximetry sensor coupled to the fiber optic cable.

12. The apparatus of claim 1, wherein the processor portion further comprises:
an accelerometer.

13. The apparatus of claim 1, wherein the processor portion comprises at least one of (i) a transceiver or (ii) a transmitter.

14. The apparatus of claim 1, further comprising:
a remote processing device configured for communication with the processor portion.

15. The apparatus of claim 1, further comprising:
a replaceable sheath configured to cover at least a portion of the first end of the conductor portion.

16. The apparatus of claim 15, further comprising:
a battery assembly coupled to the replaceable sheath.

17. The apparatus of claim 16, wherein the battery assembly comprises:
a housing having a fastener for engaging the processor portion to secure the battery assembly to the processor portion and to fix the position of the replaceable sheath on the conductor portion.

18. A method for monitoring at least one physiological parameter of an animal from one or more physiological characteristics present within an auditory canal of the animal, the animal having a head with an ear, the ear including the auditory canal, the method comprising the steps of:
sensing one or more physiological characteristics present within the auditory canal of the animal, the one or more physiological characteristics associated with at least one physiological parameter;
passing the one or more physiological characteristics through a conductor from the auditory canal to a device positioned remote from the auditory canal; and
processing the one or more sensed physiological characteristics at the device positioned remote from the auditory canal to determine the at least one physiological parameter.

19. The method of claim 18, wherein the ear further includes an auricle adjacent the auditory canal and wherein the device is positioned at least partially between the auricle of the ear and the head of the animal.

20. The method of claim 18, further comprising the step of storing at least one of (i) the one or more physiological characteristics or (ii) the determined physiological parameter.

21. The method of claim 18, wherein the sensing step comprises the step of sensing at least one of the one or more physiological characteristics from within the auditory canal of the animal and wherein the method further comprises the step of:
generating a signal corresponding to the at least one sensed physiological characteristic within the auditory canal; and
passing the signal from within the auditory canal to the device positioned remote from the auditory canal for processing.

22. The method of claim 18, wherein the sensing step comprises the step of:
passing at least one of the one or more physiological characteristics from within the auditory canal to the device positioned remote from the auditory canal for processing.

23. The method of claim 18, further comprising the step of:
communicating with a remote device;
monitoring a signal strength of communications with the remote device; and
generating an aural notification signal within the auditory canal of the animal when the signal strength is less than a predetermined value.

24. The method of claim 18, wherein the at least one physiological parameter includes temperature.

25. The method of claim 24, further comprising the steps of:
monitoring temperature at predetermined intervals for a predetermined period of time; and
detecting ovulation based on the monitored temperature.

26. The method of claim 25, wherein the at least one physiological parameter includes movement and wherein the step of detecting ovulation is further based on movement.

27. The method of claim 18, further comprising the step of:
detecting movement of the head;
wherein the processing step further processes the detected movement.

28. The method of claim 27, further comprising the steps of:
monitoring the movement of the head and the one or more physiological characteristics; and
detecting sleep apnea responsive to the monitored movement of the head and the one or more physiological characteristics.

29. The method of claim 27, further comprising the steps of:
monitoring the movement of the head; and
generating an emergency alert if the detected movement of the head exceeds a predefined value.

30. The method of claim 18, further comprising the steps of:
monitoring at least one of (i) the one or more physiological characteristics or (ii) the at least one physiological parameters; and
generating an emergency alert if one or more of the monitored physiological characteristics or parameter is outside of a predefined range for that physiological characteristic or parameter.
